(19) United States
(12) Patent Application Publication
(10) Pub. No.: US 2024/0091006 A1
(43) Pub. Date: Mar. 21, 2024

(54) IMPLANTABLE MEDICAL DEVICE WITH VISUAL ORIENTATION INDICATOR

(71) Applicant: BOSTON SCIENTIFIC SCIMED, INC., Maple Grove, MN (US)

(72) Inventors: James M. Anderson, Corcoran, MN (US); Kelsey Rae Cooper, Andover, MN (US); Joshua Stephan Havel, Champlin, MN (US); Levi Joel Wolterstorff, Saint Paul, MN (US); Shilpika Chowdhury, Minneapolis, MN (US)

(73) Assignee: BOSTON SCIENTIFIC SCIMED, INC., MAPLE GROVE, MN (US)

(21) Appl. No.: 18/469,796

(22) Filed: Sep. 19, 2023

Related U.S. Application Data
(60) Provisional application No. 63/408,210, filed on Sep. 20, 2022.

(51) Int. Cl. A61F 2/24

(52) U.S. Cl. CPC A61F 2/243; A61F 2250/0098

(57) ABSTRACT

An implantable medical device is adapted to be implanted at an implantation site within the vasculature and is capable of being implanted at the implantation site within the vasculature at more than one position relative to the implantation site. This may include a relative axial position and/or a relative rotational position. The implantable medical device includes an expandable frame that is adapted to expand from a collapsed configuration for delivery to an expanded configuration for deployment, and one or more radiopaque markers disposed relative to the expandable frame such that fluoroscopic imaging of the implantable medical device during deployment provides an indication of a position of the implantable medical device relative to the implantation site. The implantable medical device may be a replacement cardiac valve such as a replacement aortic valve, for example.

### CROSS-REFERENCE TO RELATED APPLICATIONS

This application claims the benefit of priority of U.S. Provisional Application No. 63/408,210 filed Sep. 20, 2022, the entire disclosure of which is hereby incorporated by reference.

### TECHNICAL FIELD

The present disclosure pertains to medical devices, and methods for manufacturing and using medical devices. More particularly, the disclosure is directed to implantable medical devices having a visual indicator showing appropriate orientation during and after implantation.

### BACKGROUND

A wide variety of medical devices have been developed for medical use, for example, for use in accessing body cavities and interacting with fluids and structures in body cavities. Some of these devices may include guidewires, catheters, pumps, motors, controllers, filters, grinders, needles, valves, and delivery devices and/or systems used for delivering such devices. These devices are manufactured by any one of a variety of different manufacturing methods and may be used according to any one of a variety of methods. Of the known medical devices and methods, each has certain advantages and disadvantages.

### SUMMARY

This disclosure provides design, material, manufacturing method, and use alternatives for medical devices. An example may be found in an implantable medical device adapted to be implanted at an implantation site within the vasculature, the implantable medical device capable of being implanted at the implantation site within the vasculature at more than one position relative to the implantation site. The implantable medical device includes an expandable frame that is adapted to expand from a collapsed configuration for delivery to an expanded configuration for deployment, and one or more radiopaque markers disposed relative to the expandable frame such that fluoroscopic imaging of the implantable medical device during deployment provides an indication of a position of the implantable medical device relative to the implantation site.

Alternatively or additionally, at least some of the one or more radiopaque markers may be positioned to provide an indication of an axial position of the implantable medical device relative to the implantation site via fluoroscopic imaging.

Alternatively or additionally, at least some of the one or more radiopaque markers may be positioned to provide an indication of a rotational position of the implantable medical device relative to the implantation site via fluoroscopic imaging.

Alternatively or additionally, at least some of the one or more radiopaque markers may include radiopaque marker bands that are crimped onto a portion of the expandable frame.

Alternatively or additionally, at least some of the one or more radiopaque markers may include radiopaque wires wrapped around a portion of the expandable frame.

Alternatively or additionally, at least some of the one or more radiopaque markers may include radiopaque sutures sewn around a portion of the expandable frame.

Another example may be found in a replacement cardiac valve adapted to be implanted within a native cardiac valve annulus, the replacement cardiac valve capable of being implanted in more than one position relative to the native cardiac valve annulus. The replacement cardiac valve includes an expandable frame that is adapted to expand from a collapsed configuration for delivery to an expanded configuration for deployment, and one or more radiopaque indicators disposed relative to the expandable frame such that fluoroscopic imaging of the replacement cardiac valve during deployment provides an indication of a position of the replacement cardiac valve relative to the native cardiac valve annulus. The expandable frame includes an annular portion adapted to engage the native cardiac valve annulus when deployed, a plurality of commissural posts adapted to extend above the native cardiac valve annulus when deployed, and a valve material secured relative to the plurality of commissural posts, with the valve material forming a valve cusp between each of the plurality of commissural posts.

Alternatively or additionally, at least some of the one or more radiopaque indicators may be adapted to provide an indication of insertion depth of the replacement cardiac valve relative to the native cardiac valve annulus.

Alternatively or additionally, at least some of the one or more radiopaque indicators may be secured relative to the annular portion of the expandable frame that is adapted to engage the native cardiac valve annulus when deployed.

Alternatively or additionally, at least some of the one or more radiopaque indicators may be adapted to provide an indication of relative rotational position of the replacement cardiac valve relative to the native cardiac valve annulus.

Alternatively or additionally, at least some of the one or more radiopaque indicators may be secured relative to at least some of the plurality of commissural posts.

Alternatively or additionally, the expandable frame may include a plurality of struts, and one or more of the plurality of struts are adapted to accommodate at least some of the one or more radiopaque indicators.

Alternatively or additionally, one or more of the plurality of struts may be formed with a narrowed portion adapted to accommodate a radiopaque marker band there about.

Alternatively or additionally, the expandable frame may include a plurality of loops, and one or more of the loops are adapted to accommodate one or more radiopaque indicators within at least some of the loops.

Another example may be found in an aortic valve adapted to be implanted within a native aortic valve annulus, the aortic valve capable of being implanted in more than one position relative to the native aortic valve annulus. The aortic valve includes an expandable frame that is adapted to expand from a collapsed configuration for delivery to an expanded configuration for deployment, and that includes an annular portion adapted to engage the native aortic valve annulus when deployed, a plurality of commissural posts adapted to extend above the native cardiac valve annulus when deployed, a plurality of stabilization arches adapted to extend above the plurality of commissural posts, and a valve material secured relative to the plurality of commissural posts, with the valve material forming a valve cusp between each of the plurality of commissural posts. A plurality of radiopaque indicators are disposed relative to the expandable frame such that fluoroscopic imaging of the aortic valve during deployment provides an indication of a position of the aortic valve relative to the native aortic valve annulus.

Alternatively or additionally, at least some of the plurality of radiopaque indicators may be adapted to provide an indication of insertion depth of the aortic valve relative to the native aortic valve annulus.

Alternatively or additionally, at least some of the plurality of radiopaque indicators may be secured relative to the annular portion of the expandable frame that is adapted to engage the native aortic valve annulus when deployed.

Alternatively or additionally, at least some of the plurality of radiopaque indicators may be adapted to provide an indication of relative rotational position of the aortic valve relative to the native aortic valve annulus.

Alternatively or additionally, at least some of the plurality of radiopaque indicators may be secured relative to at least some of the plurality of commissural posts.

Alternatively or additionally, at least some of the plurality of radiopaque indicators may be adapted to provide an indication of insertion depth of the aortic valve relative to the native aortic valve annulus and at least some of the plurality of radiopaque indicators are adapted to provide an indication of relative rotational position of the aortic valve relative to the native aortic valve annulus.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1A is a view of an illustrative replacement aortic valve expandable frame;

FIG. 1B is a view of an illustrative replacement aortic valve including the expandable frame of FIG. 1A;

FIG. 2A is a schematic view of an illustrative replacement aortic valve at a first axial position with respect to a native aortic valve annulus;

FIG. 2B is a schematic view of an illustrative replacement aortic valve at a second axial position with respect to a native aortic valve annulus;

FIG. 3A is a schematic view of an illustrative replacement aortic valve misaligned with respect to the native aortic valve;

FIG. 3B is a schematic view of an illustrative replacement aortic valve that is appropriately aligned with the native aortic valve;

FIG. 4 is a schematic view of an illustrative replacement aortic valve;

FIGS. 5A, 5B, 5C and 5D are views of illustrative replacement aortic valves, denoting possible locations for various radiopaque indicators or markers;

FIG. 6A is a view of an illustrative laser-cut blank for forming a replacement aortic valve, indicating features to accommodate radiopaque marker bands;

FIG. 6B is an enlarged view of a portion of FIG. 6A;

FIG. 7A is a view of an illustrative laser-cut blank for forming a replacement aortic valve, indicating features to accommodate radiopaque marker bands;

FIG. 7B is an enlarged view of a portion of FIG. 7A;

FIG. 8 is a view of an illustrative laser-cut blank for forming a replacement aortic valve;

FIG. 9A is a schematic cross-sectional view of an illustrative radiopaque pin usable with the laser-cut blank of FIG. 8;

FIG. 9B is a side view of an illustrative radiopaque pin and suturing relative to the laser-cut blank of FIG. 8; and

FIGS. 10A and 10B are views of illustrative replacement aortic valves including radiopaque sutures.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

A number of implantable medical devices are implanted at a variety of different implantation sites within a patient. In some cases, some implantable medical devices are capable of being implanted in more than one possible position at a particular implantation site. For example, some implantable medical devices, by basis of their configuration or overall shape, may be capable of being implanted in more than one axial position relative to the implantation site. If the implantation site is an annulus, for example, the implantable medical device may be capable of being implanted at differing penetration depths relative to the annulus. Some anatomies and/or physician's preferences may dictate a more proximal implantation position, or relatively less penetration depth. Some anatomies and/or physician's preferences may dictate a more distal implantation position, or a relatively greater penetration depth.

Some implantable medical devices, by basis of their configuration or overall shape, may be capable of being implanted in more than one rotational position relative to the implantation site. In some cases, an implantable medical device may be capable of being implanted at more than one axial position and more than one rotational position relative to the implantation site. With respect to rotational position, which may refer to a relative rotational orientation of the implantable medical device, the implantable medical device may be implanted such that a particular reference point on the implantable medical device, for example, may be facing any particular rotational point as defined along a 360 degree circle. The reference point may be facing a direction of 45 degrees, or perhaps 310 degrees, or any of a variety of different directions. For some implantable medical devices, the rotational position or orientation may not matter. For some implantable medical devices, the rotational position or orientation may be important.

For ease of illustration, the disclosure is directed to the implantable medical device being a replacement cardiac valve, such as a replacement aortic valve, that is deliverable in a trans-catheter manner. However, the disclosure is intended to not be so limited, as the replacement aortic valve described herein is merely illustrative.

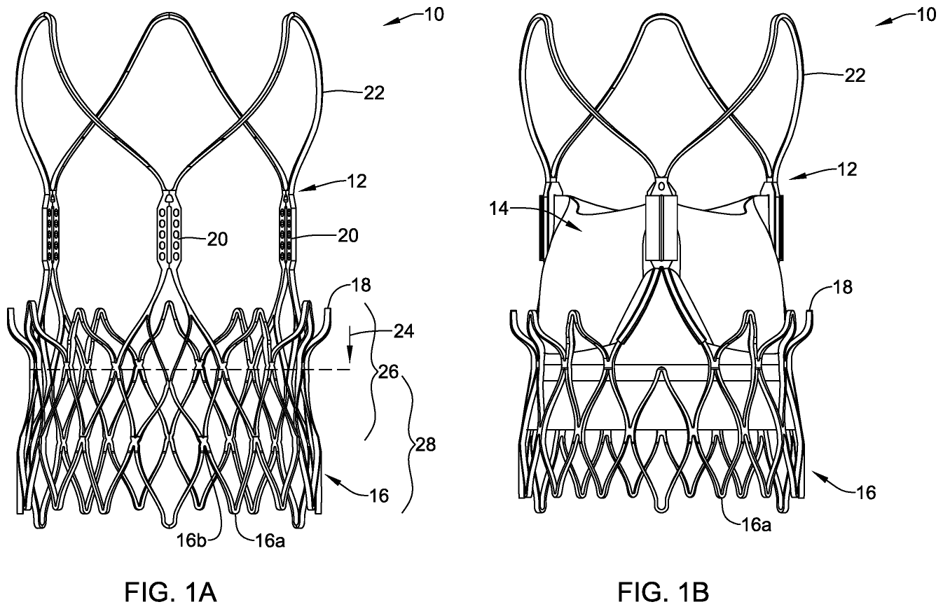

FIG. 1A and FIG. 1B are side views of an illustrative replacement cardiac valve 10. The replacement cardiac valve 10 may be a replacement aortic valve, a replacement mitral valve, a replacement pulmonary valve or a replacement tricuspid valve, for example. In some cases, the replacement cardiac valve 10 may include biological tissue such as porcine or bovine pericardium and/or natural cardiac valve leaflets such as natural porcine cardiac valve leaflets. In some cases, the natural cardiac valve leaflets may be attached to a portion of natural cardiac wall tissue. The biological material may be fixed, for example, using glutaraldehyde.

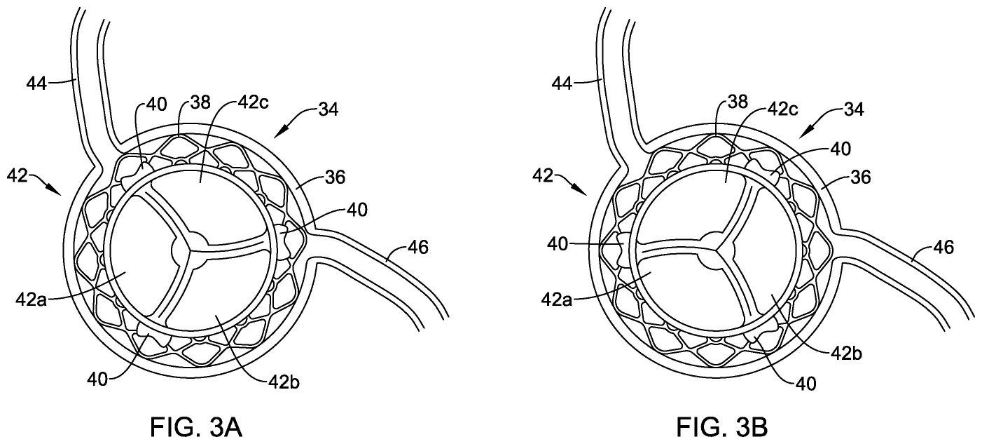

The replacement cardiac valve 10 includes an expandable frame 12 that may be compressible to a radially compressed, or collapsed, configuration for delivery using a delivery catheter, and may be expandable to an expanded configuration (as shown) during implantation. The replacement cardiac valve 10 may include a plurality of leaflets defining a valve 14 (as seen in FIG. 1B), the position of which is depicted schematically by the bounding phantom lines. The leaflets defining the valve 14 are visible in FIGS. 3A and 3B (including leaflets 42a, 42b and 42c), for example.

In some cases, the expandable frame may include a lower tubular or crown portion 16, an upper crown portion 18, a plurality of upstanding commissural posts 20, and a plurality of stabilization arches 22. In use, the lower portion 16 of the expandable frame 12 may be adapted to be deployed after the other regions of the expandable frame 12. For example, the arches 22, the supports 20 and the upper crown 18 may be deployed at least partly before the lower portion 16 (in that order, or in reverse order, or in a different order). At the very least, once the upper crown 18 has been at least partly deployed, the expandable frame 12 may be urged and/or displaced in the direction of arrow 24 to seat the upper crown 18 against native leaflets at the implantation site. Deploying the lower portion 16 last fixes the expandable frame 12 in its final position.

The lower portion 16, and optionally a portion of the upper crown 18, may be formed by a lattice structure of the stent. The lattice structure may define cells or apertures, for example, generally diamond-shaped apertures. In some cases, the native leaflets may generally overlap a portion 26 of the expandable frame 12. The native valve annulus may overlap a portion 28 of the expandable frame. In some instances, the lower portion 16 may have an extremity formed with a substantially zig-zag shape. The zig-zag shape may include lower apexes 16a and upper apexes 16b. The upper apexes 16b may be masked in FIG. 1 by the superimposed presentation of both the frontmost and rearmost cells of the lattice structure. The zig-zag shape may be substantially continuous around the circumference of the expandable frame 12.

The expandable frame 12 may optionally be of a self-expanding type that is compressible to the compressed configuration for loading into a delivery catheter for delivery to the site of implantation. In use, by removal of the constraining effect of a sheath holding the expandable frame 12 in the compressed configuration, the expandable frame 12 self-expands to or towards the operative configuration. A self-expanding stent may, for example, be of shape-memory material, for example, shape-memory metal alloy, for example, nitinol. Alternatively, the expandable frame 12 may be configured to be expanded by application of a foreshortening force from the delivery catheter and/or by application of expanding force from the delivery catheter, such as by using an expansion balloon. These are just examples.

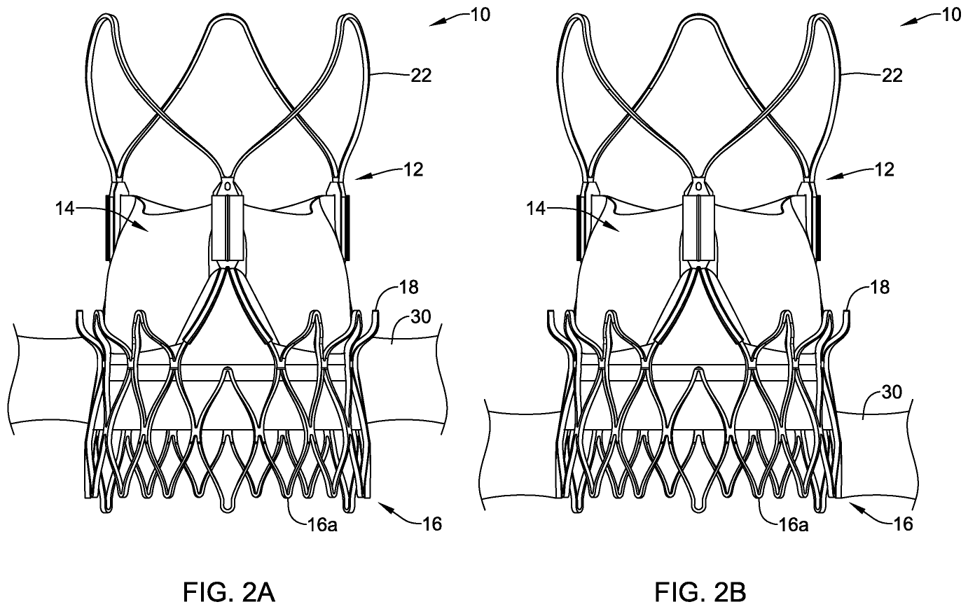

FIG. 2A shows a schematic view of the replacement cardiac valve 10 that has been implanted within a native cardiac annulus 30. It will be appreciated that the native cardiac annulus 30 is shown schematically, and for ease of illustration, the native cardiac valve cusps are not shown. In FIG. 2A, it can be seen that the replacement cardiac valve 10 has been implanted, or at least positioned, at a first axial position, or penetration depth, relative to the native cardiac annulus 30. In FIG. 2B, it can be seen that the replacement cardiac valve 10 has been implanted, or at least positioned, at a second axial position, or penetration depth, relative to the native cardiac annulus 30. In some cases, the replacement cardiac valve 10 may include a tissue skirt (not shown) that extends down (in the illustrated orientation) to the lower apexes 16a, for example.

In some cases, a physician may have a preference as to the axial position, or penetration depth, that they prefer for implantation of the replacement cardiac valve 10. In some instances, unique features of the patient's anatomy, such as but not limited to the particular anatomy of the patient's native cardiac annulus 30, or the sizing of the native valve cusps that are pushed to the side by the replacement cardiac valve 10, may suggest a particular axial position or penetration depth into the native cardiac annulus 30. Particular features of the patient's vasculature may dictate an optimal positioning for the stabilization arches 22. Any of a variety of different anatomical features may dictate an optimal axial position for the replacement cardiac valve 10 for a particular patient. As will be discussed, the replacement cardiac valve 10 may include features that allow for easy identification of the axial position, or penetration depth, of the replacement cardiac valve 10 during implantation. In some instances, the replacement cardiac valve 10 may include features that are visible via fluoroscopy.

In some cases, the relative rotational position or orientation of the replacement cardiac valve 10 may be important, particularly when the replacement cardiac valve 10 is a replacement aortic valve, intended for implantation within a native aortic annulus. In some cases, portions of the replacement cardiac valve 10 may potentially interfere with subsequent access to the coronary arteries. In some patients, particularly younger patients, there may be a subsequent need to implant a new replacement aortic valve some years after the initial implantation. In some patients, there may be a need, either immediate or down the road, to access one or more of the coronary arteries in order to perform angioplasty or rotational atherectomy, for example. There may be a need to access one or more of the coronary arteries in order to implant one or more stents. If the initially implanted replacement aortic valve interferes with access to the coronary arteries, this can be problematic.

FIG. 3A shows a schematic view of a replacement aortic valve 34 that has been implanted within a native aortic annulus 36. The replacement aortic valve 34 may be considered as being an example of the replacement cardiac valve 10 shown and described in FIGS. 1A and 1B. The replacement aortic valve 34 includes an expandable frame 38 that may be similar to the expandable frame 12. The expandable frame 38 includes several commissure posts 40. In some case, valve leaflets 42, individually labeled as 42a, 42b and 42c, are secured relative to the commissure posts 40. As shown, a coronary artery 44 and a coronary artery 46 are each connected to the native aortic annulus 36.

FIG. 3A illustrates a poor rotational orientation of the replacement aortic valve 34, as it can be seen that one of the commissure posts 40 is at least partially blocking the coronary artery 44 and another one of the commissure posts 40 is at least partially blocking the coronary artery 46. While both coronary arteries 44 and 46 are shown as being partially blocked, in some cases the particular location of the coronary arteries 44 and 46 in some patients may mean that a poor rotational orientation of the replacement aortic valve 34 may result in only one of the coronary arteries 44 and 46 being at least partially blocked.

FIG. 3B illustrates an optimal rotation orientation of the replacement aortic valve 34, as it can be seen that none of the commissure posts 40 are blocking the coronary artery 44 and none of the commissure posts 40 are blocking the coronary artery 46. As a result of this rotational orientation, the replacement aortic valve 34 will not cause problems, or at least should cause fewer problems, if the need arises in the future to implant a second replacement aortic valve. This also means that the coronary arteries 44 and 46 are easily reachable for any ensuing procedures within the coronary arteries 44 and 46, such as but not limited to angioplasty, rotational atherectomy or stent implantation. As will be discussed, the replacement aortic valve 34 may include features that allow for easy identification of the rotational orientation or position of the replacement aortic valve 34 during implantation. In some instances, the replacement aortic valve 34 may include features that are visible via fluoroscopy.

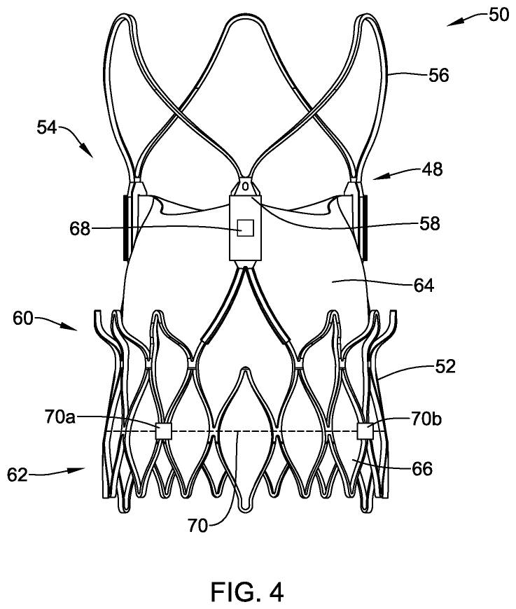

As noted, implantable medical devices such as replacement cardiac valves may include features that allow fluoroscopic viewing of the relative axial and/or rotational positioning of the implantable medical devices. FIG. 4 is a view of an illustrative replacement aortic valve 48 shown disposed within an aorta 50, with the replacement aortic valve 48 engaging the native aortic annulus 52. The replacement aortic valve 48 includes an expandable frame 54 including stabilization arches 56, commissural posts 58, an upper crown portion 60 and a lower crown portion 62. The replacement aortic valve 48 includes a tissue valve 64 that is supported by the expandable frame 54 as well as a tissue skirt 66.

FIG. 4 shows possible locations for where radiopaque indicators or markers may be located. In some cases, when there is a desire to be able to fluoroscopically ascertain a relative rotational orientation or positioning of the replacement aortic valve 48, radiopaque indicators or markers may be secured relative to the commissural posts 58, at location 68. In some cases, where there is a desire to be able to fluoroscopically ascertain a relative axial positioning, or penetration depth relative to the native aortic annulus 52, radiopaque indicators or markers may be secured relative to the replacement aortic valve 48 along a line 70, such as at a location 70a and a location 70b. There may be one or more additional radiopaque indicators or markers disposed along a back side (not visible in this orientation) of the replacement aortic valve 48.

In some cases, the radiopaque indicators or markers that are disposed along the line 70 may be vertically aligned with the commissural posts 58, for example, in which case the same radiopaque indicators or markers may indicate both axial positioning and rotational positioning of the replacement aortic valve 48 relative to the native aortic annulus 52. The replacement aortic valve 48 may be rotated, if necessary, for the radiopaque indicators or markers to appropriately align relative to the native aortic annulus 52 such that the expandable frame 54 does not interfere with access to the coronary arteries 44 and 46 (FIGS. 3A and 3B). Because the radiopaque indicators or markers are also positioned along the line 70, the same radiopaque indicators or markers can also indicate, under fluoroscopy, the relative axial position of the replacement aortic valve 48 relative to the native aortic annulus 52.

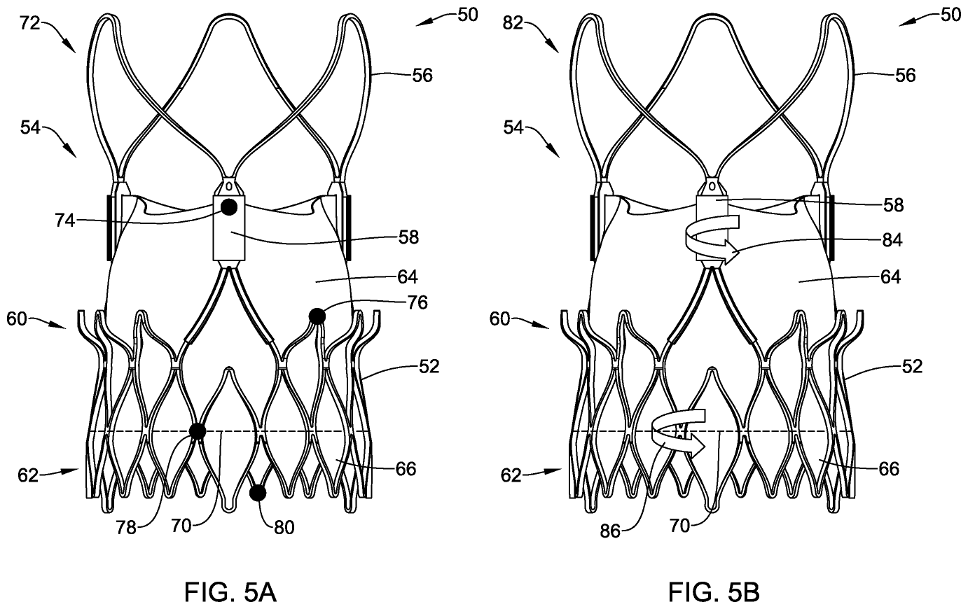

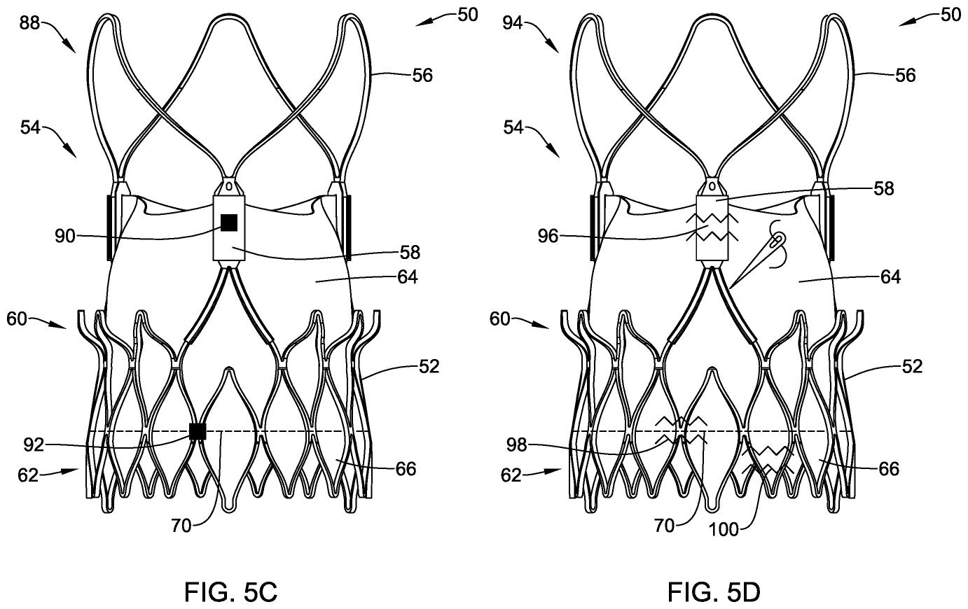

FIGS. 5A, 5B, 5C and 5D each show illustrative replacement aortic valves with radiopaque indicators or markers added using varying techniques. FIG. 5A shows an illustrative replacement aortic valve 72 that is similar to the replacement aortic valve 48. The replacement aortic valve 72 includes loops that are added with marker band pucks. In some cases, loops may be formed of wire or composite thread that is applied as a single element with ends fused or terminated together either thermally or mechanically to form a loop. In some cases, the loop ends may terminate within a puck or band. The marker band pucks may be formed of a radiopaque material such as but not limited to gold or tantalum, for example. A loop with marker band puck 74 is shown secured relative to a commissural post 58. A loop with marker band puck 76 is shown secured relative to the upper crown portion 60. A loop with marker band puck 78 is shown secured relative to the expandable frame 54 along the line 70. A loop with marker band 80 is shown secured relative to the lower crown portion 62. Marker bands may be open ended crimped or swaged onto mating geometry, but may remain open-ended radially. It will be appreciated that a loop with marker band may be secured to any of a variety of different locations about the replacement aortic valve 72, depending on what positioning features are desired to control.

FIG. 5B shows an illustrative replacement aortic valve 82 that is similar to the replacement aortic valve 48. The replacement aortic valve 82 includes radiopaque wire that is wound onto the expandable frame 54, or perhaps the tissue forming either the tissue valve 64 or the tissue skirt 66. The radiopaque wire may be formed of gold or tantalum, for example. As shown, a radiopaque indicator may be formed at a location 84 by winding a radiopaque wire around the commissural post 58. In some cases, while only one of the commissural posts 58 is shown as including a radiopaque wire wound about the commissural post 58, in some cases each of the commissural posts 58 may include a radiopaque wire wound about each commissural post 58. The replacement aortic valve 82 includes a radiopaque indicator that is formed at a location 86, which is located along the line 70, by wrapping a radiopaque wire around part of the expandable frame 54.

FIG. 5C shows an illustrative replacement aortic valve 88 that is similar to the replacement aortic valve 48. The replacement aortic valve 88 includes a radiopaque marker band that is disposed at a location 90 that is proximate one of the commissural posts 58. While only one commissural post 58 is shown as including a radiopaque marker band, it will be appreciated that in some cases each of the three commissural posts 58 may include a radiopaque marker band. The marker bands may be formed of gold or tantalum, for example. The replacement aortic valve 88 includes a radiopaque marker band that is disposed at a location 92 that is located along the line 70. While one radiopaque marker band is shown along the line 70, in some cases the replacement aortic valve 88 may include a plurality of radiopaque marker bands along the line 70. While not shown in this fashion, the radiopaque marker bands along the line 70 may be aligned with the commissure posts 58 in order to provide both axial and rotational positioning of the replacement aortic valve 88 relative to the native aortic annulus 52.

FIG. 5D shows an illustrative replacement aortic valve 94 that is similar to the replacement aortic valve 48. The replacement aortic valve 94 includes a radiopaque suture or film at a location 96 relative to the commissural post 58. The radiopaque suture or film may be secured to the tissue valve 64 or to the commissural post 58 itself. While only one commissural post 58 is shown as including the radiopaque suture or film, in some cases each of the three commissural posts 58 may include the radiopaque suture or film. The replacement aortic valve 94 includes a radiopaque suture or film at a location 98 that is disposed along the line 70. The replacement aortic valve 94 includes a radiopaque suture or film at a location 100 that is located along the lower crown portion 62.

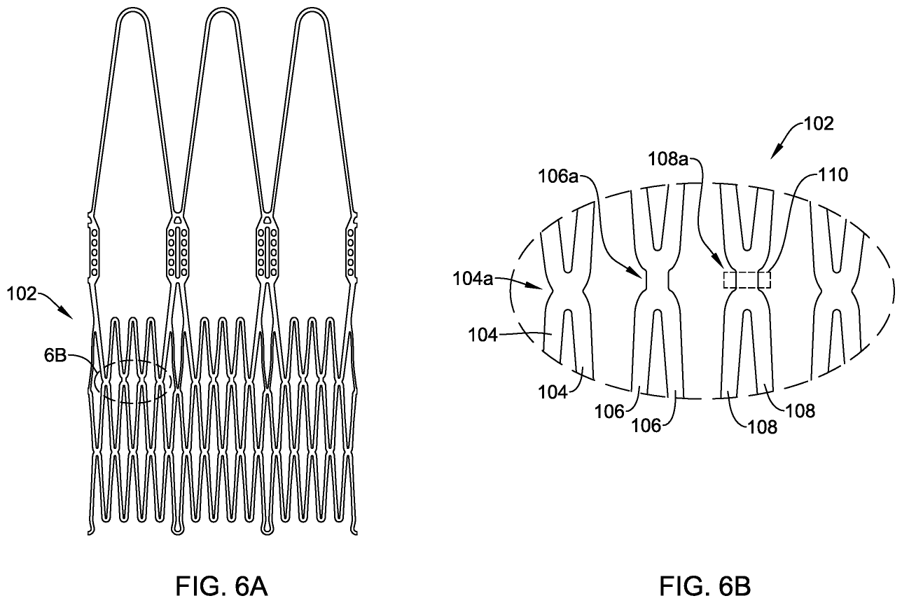

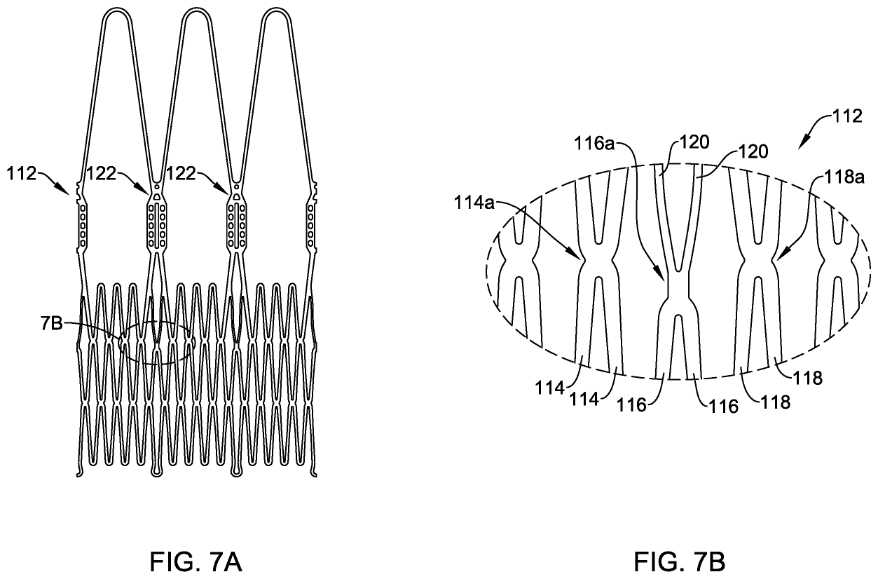

The expandable frames 12, 38 and 54 may be formed in a variety of ways. In some cases, the expandable frames 12, 38 and 54 may be laser-cut from a Nitinol tube, for example. FIG. 6A shows an illustrative laser-cut pattern 102 that may be used to create one of the expandable frames described herein. The laser-cut pattern 102 is shown schematically in a two-dimensional manner, even though the laser-cut tube is circular in cross-section. A portion of the laser-cut pattern 102 has been enlarged in FIG. 6B to better show features of the laser-cut pattern 102 that allow radiopaque marker bands to be secured to the laser-cut pattern 102. As can be seen, the laser-cut pattern 102 includes a number of “X” shapes in which pairs of struts 104, 106 and 108 come together before separating once again. The pair of struts 104 come together in a crossing region 104a having a first profile. The pair of struts 106 come together in a crossing region 106a having a second profile. The pair of struts 108 come together in a crossing region 108a having the same second profile. The second profile includes a narrowing that allows a marker band, such as a marker band 110 shown in phantom, to be crimped about the crossing region. In some cases, as shown, the crossing regions 104a, 106a and 108a may be aligned in a row. In some cases, the crossing regions 104a, 106a and 108a may be misaligned, with some above and some below a midpoint defined therebetween. The marker band 110 may be formed of any radiopaque material such as gold or tantalum, for example.

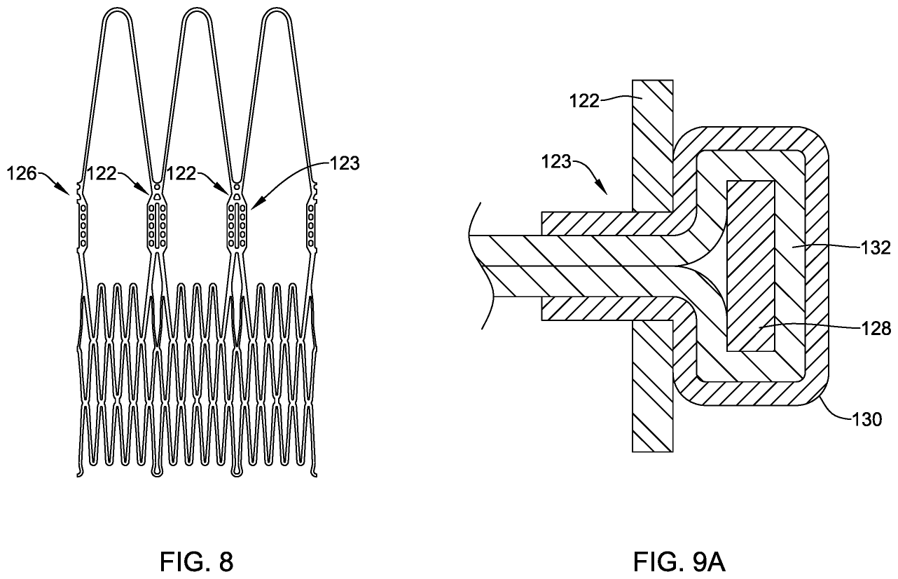

FIG. 7A shows an illustrative laser-cut pattern 112 that may be used to create one of the expandable frames described herein. The laser-cut pattern 112 is shown schematically in a two-dimensional manner, even though the laser-cut tube is circular in cross-section. A portion of the laser-cut pattern 112 has been enlarged in FIG. 7B to better show features of the laser-cut pattern 112 that allow radiopaque marker bands to be secured to the laser-cut pattern 112. As can be seen, the laser-cut pattern 112 includes a number of “X” shapes in which pairs of struts 114, 116 and 118 come together before separating once again. The pair of struts 114 come together in a crossing region 114a having a first profile. The pair of struts 116 come together in a crossing region 116a having a second profile. In some instances, the pair of struts 116 include a region 120 that extends upwardly (in the illustrated orientation) from the crossing region 116a and is adapted to not bend, comply or contribute to implant radial expansion. The pair of struts 118 come together in a crossing region 118a having the same first profile. The second profile includes a narrowing that allows a marker band to be crimped about the crossing region. In some cases, the crossing regions 114a, 116a and 118a may be aligned in a row. In some cases, as shown, the crossing regions 114a, 116a and 118a may be misaligned, with some above and some below a midpoint defined therebetween. In some cases, an added length of the crossing region 116a may improve strain relief distribution. The marker bands may be formed of any radiopaque material such as gold or tantalum, for example. In some cases, the marker bands may be welded or swaged into position relative to the laser-cut pattern 112.

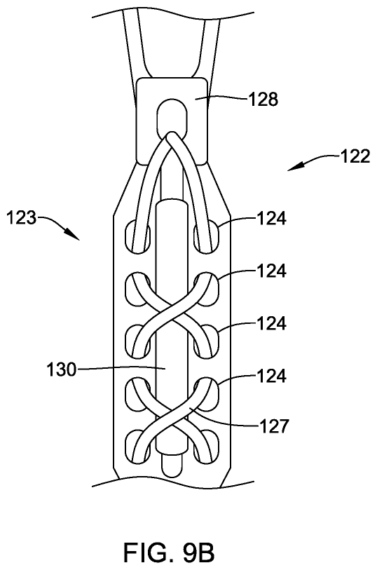

FIG. 8 shows an illustrative laser-cut pattern 126 that may be used to create one of the expandable frames described herein. The laser-cut pattern 112 is shown schematically in a two-dimensional manner, even though the laser-cut tube is circular in cross-section. The laser-cut pattern 112 may include a slot 123 that is formed within each of the commissural posts 122. In some cases, the slots 123 may be adapted to permit tissue or other material to be pulled through the slot 123, and then a radiopaque pin may be inserted through the material to hold the material relative to the slot 123. This may be seen for example in FIGS. 9A and 9B.

FIG. 9A shows a radiopaque pin 128 that may used in combination with one of the slots 123 to help hold tissue or other material in place. FIG. 9A may be considered as a top view, as it is a top of the radiopaque pin 128 that is visible. As seen in FIG. 9A, a membrane 132 (which may for example represent tissue forming part of the valve 14) has been pulled through the slot 123, formed in the commissural post 122. In some cases, as shown, the membrane 132 is at least partially surrounded by a PET woven fabric 130. Bn inserting the radiopaque pin 128, the membrane 132 and the PET woven fabric 130 are held in place relative to the commissural post 122. Because the radiopaque pin 128 is coated with or formed from a radiopaque material, the radiopaque pin 128 will be visible during fluoroscopy. The radiopaque pin 128 may be laser cut and polished, stamped, machined, or 3D printed.

FIG. 9B is an enlarged side view of a commissural post 122 showing the PET woven fabric 130 pulled through the slot 123. The radiopaque pin 128 has been inserted into a space formed by the PET woven fabric 130, thereby preventing the PET woven fabric 130 from pulling back through the slot 123. While not shown, since the PET woven fabric 130 is on the outside, the membrane 132 similarly extends through the slot 123 and is held in position by the radiopaque pin 128. As can be seen, the commissural post 122 includes a number of apertures 124 through which a suture 127 extends. The suture 127 helps to hold everything together. In some cases, the suture 127 may include or be formed of a radiopaque material such that the suture 127 is visible during fluoroscopy. The suture 127 may include or be formed of materials such as polyester, polyurethane, nylon, silk or collagen and may be coated with a radiopaque material such as platinum, tantalum, iridium, carbon, gold, graphene and tungsten, as well as composites thereof. The suture 127 may be coated with a radiopaque material such as barium, iodine, bismuth, tungsten or combinations thereof. In some cases, the radiopaque coating on the suture 127 may include one or more radiopaque materials disposed within a coating material.

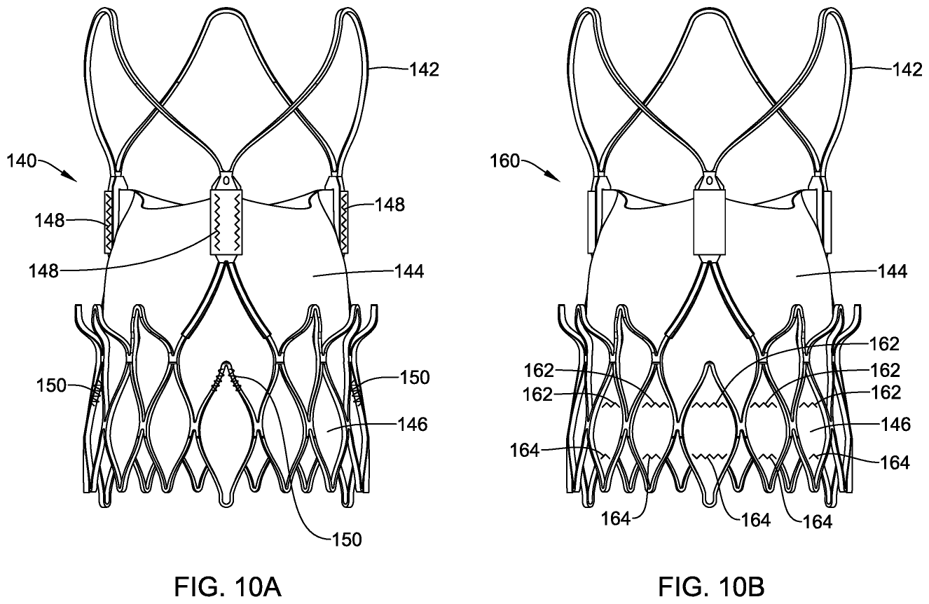

In some cases, radiopaque sutures may be provided in or on other parts of a replacement aortic valve in order to provide radiopaque indicators. FIGS. 10A and 10B show illustrative but non-limiting examples of illustrative replacement aortic valves that include radiopaque sutures as radiopaque indicators. FIG. 10A provides an example of using radiopaque sutures to provide for commissural alignment (rotational orientation or position) while FIG. 10B provides several examples of using radiopaque sutures to provide for annulus alignment (axial position or penetration depth).

FIG. 10A is a view of an illustrative replacement aortic valve 140 that includes an expandable frame 142 supporting a tissue valve 144 and a tissue skirt 146. The replacement aortic valve 140 includes suture patterns 148 that are aligned with each of the commissural posts formed within the expandable frame 142. Alternatively or additionally, the replacement aortic valve 140 also includes suture patterns 150 that are vertically aligned with the commissural posts, but are located lower, closer to where the replacement aortic valve 140 will contact a native aortic annulus upon deployment. The suture patterns 148 and 150 may be formed by suturing into the tissue, for example, or by wrapping the radiopaque suture material around the expandable frame 142 itself.

FIG. 10B is a view of an illustrative replacement aortic valve 160 that includes the expandable frame 142 supporting the tissue valve 144 and the tissue skirt 146. The replacement aortic valve 160 includes a first suture pattern 162 that extends circumferentially about the replacement aortic valve 160 at a first axial position and a second suture pattern 164 that extends circumferentially about the replacement aortic valve 160 at a second axial position that is axially offset from the first axial position. It will be appreciated that the replacement aortic valve 160 may include the first suture pattern 162 without the second suture pattern 164. The replacement aortic valve 160 may include the second suture pattern 164 without the first suture pattern 162, depending on what part of the valve is desired to be fluoroscopically visible.

The devices described herein, as well as various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An implantable medical device adapted to be implanted at an implantation site within the vasculature, the implantable medical device capable of being implanted at the implantation site within the vasculature at more than one position relative to the implantation site, the implantable medical device comprising:
an expandable frame that is adapted to expand from a collapsed configuration for delivery to an expanded configuration for deployment; and
one or more radiopaque markers disposed relative to the expandable frame such that fluoroscopic imaging of the implantable medical device during deployment provides an indication of a position of the implantable medical device relative to the implantation site.

2. The implantable medical device of claim 1, wherein at least some of the one or more radiopaque markers are positioned to provide an indication of an axial position of the implantable medical device relative to the implantation site via fluoroscopic imaging.

3. The implantable medical device of claim 1, wherein at least some of the one or more radiopaque markers are positioned to provide an indication of a rotational position of the implantable medical device relative to the implantation site via fluoroscopic imaging.

4. The implantable medical device of claim 1, wherein at least some of the one or more radiopaque markers comprise radiopaque marker bands that are crimped onto a portion of the expandable frame.

5. The implantable medical device of claim 1, wherein at least some of the one or more radiopaque markers comprise radiopaque wires wrapped around a portion of the expandable frame.

6. The implantable medical device of claim 1, wherein at least some of the one or more radiopaque markers comprise radiopaque sutures sewn around a portion of the expandable frame.

7. A replacement cardiac valve adapted to be implanted within a native cardiac valve annulus, the replacement cardiac valve capable of being implanted in more than one position relative to the native cardiac valve annulus, the replacement cardiac valve comprising:
an expandable frame that is adapted to expand from a collapsed configuration for delivery to an expanded configuration for deployment, the expandable frame including
an annular portion adapted to engage the native cardiac valve annulus when deployed;
a plurality of commissural posts adapted to extend above the native cardiac valve annulus when deployed; and
a valve material secured relative to the plurality of commissural posts, with the valve material forming a valve cusp between each of the plurality of commissural posts; and
one or more radiopaque indicators disposed relative to the expandable frame such that fluoroscopic imaging of the replacement cardiac valve during deployment provides an indication of a position of the replacement cardiac valve relative to the native cardiac valve annulus.

8. The replacement cardiac valve of claim 7, wherein at least some of the one or more radiopaque indicators are adapted to provide an indication of insertion depth of the replacement cardiac valve relative to the native cardiac valve annulus.

9. The replacement cardiac valve of claim 8, wherein at least some of the one or more radiopaque indicators are secured relative to the annular portion of the expandable frame that is adapted to engage the native cardiac valve annulus when deployed.

10. The replacement cardiac valve of claim 7, wherein at least some of the one or more radiopaque indicators are adapted to provide an indication of relative rotational position of the replacement cardiac valve relative to the native cardiac valve annulus.

11. The replacement cardiac valve of claim 10, wherein at least some of the one or more radiopaque indicators are secured relative to at least some of the plurality of commissural posts.

12. The replacement cardiac valve of claim 7, wherein the expandable frame comprises a plurality of struts, and one or more of the plurality of struts are adapted to accommodate at least some of the one or more radiopaque indicators.

13. The replacement cardiac valve of claim 12, wherein one or more of the plurality of struts are formed with a narrowed portion adapted to accommodate a radiopaque marker band there about.

14. The replacement cardiac valve of claim 7, wherein the expandable frame comprises a plurality of loops, and one or more of the loops are adapted to accommodate one or more radiopaque indicators within at least some of the loops.

15. An aortic valve adapted to be implanted within a native aortic valve annulus, the aortic valve capable of being implanted in more than one position relative to the native aortic valve annulus, the aortic valve comprising:
an expandable frame that is adapted to expand from a collapsed configuration for delivery to an expanded configuration for deployment, the expandable frame including
an annular portion adapted to engage the native aortic valve annulus when deployed;
a plurality of commissural posts adapted to extend above the native cardiac valve annulus when deployed;
a plurality of stabilization arches adapted to extend above the plurality of commissural posts; and
a valve material secured relative to the plurality of commissural posts, with the valve material forming a valve cusp between each of the plurality of commissural posts; and
a plurality of radiopaque indicators disposed relative to the expandable frame such that fluoroscopic imaging of the aortic valve during deployment provides an indication of a position of the aortic valve relative to the native aortic valve annulus.

16. The aortic valve of claim 15, wherein at least some of the plurality of radiopaque indicators are adapted to provide an indication of insertion depth of the aortic valve relative to the native aortic valve annulus.

17. The aortic valve of claim 16, wherein at least some of the plurality of radiopaque indicators are secured relative to the annular portion of the expandable frame that is adapted to engage the native aortic valve annulus when deployed.

18. The aortic valve of claim 15, wherein at least some of the plurality of radiopaque indicators are adapted to provide an indication of relative rotational position of the aortic valve relative to the native aortic valve annulus.

19. The aortic valve of claim 18, wherein at least some of the plurality of radiopaque indicators are secured relative to at least some of the plurality of commissural posts.

20. The aortic valve of claim 15, wherein at least some of the plurality of radiopaque indicators are adapted to provide an indication of insertion depth of the aortic valve relative to the native aortic valve annulus and at least some of the plurality of radiopaque indicators are adapted to provide an indication of relative rotational position of the aortic valve relative to the native aortic valve annulus.
